(19) United States
(12) Patent Application Publication
(10) Pub. No.: US 2018/0326108 A1
(43) Pub. Date: Nov. 15, 2018

(54) DEODORANT TREATMENT MATERIAL FOR TOBACCO

(71) Applicant: JOHOKIKIHANBAI CO., LTD., Kagoshima (JP)

(72) Inventor: Shozo NISHIKI, Kagoshima (JP)

(73) Assignee: JOHOKIKHANBAI CO., LTD., Kagoshima-shi, Kagoshima (JP)

(21) Appl. No.: 15/762,428

(22) PCT Filed: Dec. 1, 2016
(86) PCT No.: PCT/JP2016/085770

§ 371 Date: Mar. 22, 2018

(30) Foreign Application Priority Data
Feb. 3, 2016 (JP) 2016-018888
Jun. 24, 2016 (JP) 2016-125684

(51) Int. Cl. A61L 9/012; A61L 9/013; A61L 9/014; C12P 1/02

(52) U.S. Cl. CPC A61L 9/012; A61L 9/013; C12N 1/14; C12P 1/02; A61L 9/014

(57) ABSTRACT

A deodorant treatment material for tobacco 1 includes: a highly adsorptive carbon raw material 2 which contains bamboo charcoal; a microbial raw material 3 which can generate an organic acid and which is formed with a koji mold; starches 4 whose weight ratio R with respect to the microbial raw material 3 is 0.1 to 1.2, and which serve as a nutrient source for the microbial raw material 3; and a cylindrical member 12 which makes the outside communicate with the carbon raw material and which makes the outside communicate with the microbial raw material. The starches 4 have a starch ratio of 0.1 to 0.5, the microbial raw material 3 contains at least one of a rice koji and a wheat koji and the cylindrical member 12 is formed with: a long pipe 13; and a short pipe 14 which is shorter than the long pipe 13.

### CROSS-REFERENCE TO RELATED APPLICATION

This application claims the benefit of Japan Application No. 2016-018888, filed on Feb. 3, 2016 and Application No. 2016-125684 filed on Jun. 24, 2016 with the Japanese Patent Office, the disclosures of which are incorporated herein by reference.

### BACKGROUND

### Field of the Invention

The present invention relates to a deodorant treatment material for tobacco which is used for decomposing and removing malodorous substances generated by tobacco and the combustion of tobacco. Specifically, the present invention relates to a deodorant treatment material for tobacco which simultaneously has an excellent deodorant effect on a plurality of malodorous substances generated from tobacco although the deodorant treatment material for tobacco has a simple composition.

### Background Art

Conventionally, it is known that a carbon-based raw material (hereinafter referred to as “carbon raw material”), such as bamboo charcoal or charcoal, in which a large number of micropores are formed by burning is used, and that by the large surface area thereof, it is possible to physically adsorb various malodorous components so as to remove them.

However, since this physical adsorption mainly occurs on a solid surface, when the entire solid surface is covered with the malodorous components, the adsorption capacity is significantly lowered. Hence, in order to maintain a high adsorption capacity, it is necessary to constantly replace a carbon material or perform regeneration by high-temperature heating.

A technology related to a deodorant treatment material is also known in which a specific single mold or a mold group formed with a plurality of molds is adsorbed to a carbon raw material, in which this is used as a culture medium and in which the molds are cultured and grown (see, for example, Patent Literature 1). It can be considered that in this technology, a neutralization reaction occurs which neutralizes highly alkaline malodorous components such as ammonia with various types of organic acids generated when the molds are grown and that chemical adsorption involving such a chemical reaction occurs simultaneously with the physical adsorption described above so as to further enhance a deodorant effect caused by the carbon raw material.

### CITATION LIST

### Patent Literature

Patent Literature 1: Japanese Unexamined Patent Application Publication No. H5-309385

### SUMMARY OF THE INVENTION

### Technical Problem

However, since in the deodorant treatment material disclosed in Patent Literature 1, since a nutrient source necessary for growing the molds is not contained, when the deodorant treatment material is used for a long period of time, the growth rate of the molds is lowered, and thus the amount of organic acids generated is significantly reduced, with the result that it is impossible to maintain a high deodorant effect.

A malodor specific to tobacco which is generated by smoking is mainly caused by a plurality of malodorous components such as ammonia, hydrogen sulfide, acetaldehyde, formaldehyde, acetic acid and pyridine, and thus it is necessary to simultaneously remove all the malodorous components, with the result that it is significantly difficult to effectively deodorize tobacco. In particular, a deodorant treatment material for tobacco is highly demanded which has a high ability to simultaneously remove ammonia which releases the strongest malodor and carcinogenic acetaldehyde and formaldehyde.

The present invention is made in view of the foregoing points, and has an object to provide a deodorant treatment material for tobacco which simultaneously has an excellent deodorant effect on a plurality of malodorous substances generated from tobacco although the deodorant treatment material for tobacco has a simple composition.

### Solution to Problem

In order to achieve the above object, a deodorant treatment material for tobacco according to the present invention includes: a highly adsorptive carbon raw material which contains bamboo charcoal; a microbial raw material which can generate an organic acid and which is formed with a koji mold; starches whose weight ratio with respect to the microbial raw material is 0.1 to 1.2, and which serve as a nutrient source for the microbial raw material; and a cylindrical member which makes the outside communicate with the carbon raw material and which makes the outside communicate with the microbial raw material.

For the bamboo charcoal serving as the carbon raw material, bamboo is used as the raw material, and as compared with charcoal, the diameter of a pore is small, and the surface area is large, with the result that the physical adsorption capacity is satisfactory.

Furthermore, the koji mold serving as the microbial raw material refers to a filamentous fungus of aspergillus genus which makes a koji, and is used in the form of a seed koji obtained by separately culturing the spore thereof, a rice koji obtained by breeding the koji mold in steamed rice, a wheat koji obtained by breeding the koji mold in steamed wheat, etc.

The koji mold secretes, from the tip ends of hyphae, various enzymes for decomposing starches, proteins, etc., and uses generated glucose and amino acids as nutrient sources so as to grow. Since in particular, as an enzyme, a large amount of amylase for hydrolyzing starches is secreted, when the koji is added to the starches, the saccharification of starches proceeds, and thus sugar is generated. At the same time, organic acids such as citric acid, succinic acid and malic acid are generated, and in particular, among them, a large amount of citric acid is generated. In this way, as described above, the neutralization reaction occurs on alkaline malodorous components, such as ammonia, which are the main malodorous components of tobacco, with the result that it is possible to obtain the remarkable deodorant effect.

In addition, the starches refer to natural starches, such as a potato starch, a dogtooth violet starch, a rice starch, a wheat starch and a maize starch, which are collected from grains without being processed or processed starches which are obtained, for example, by adjusting the viscosity of the natural starches. One type or a combination of two or more types may be used.

Since as described above, the koji mold can grow by using the starches as the nutrient source, the starches are contained in advance, and thus even when the period of use is long, it is possible to reliably prevent the amount of organic acids generated from being reduced as a result of a decrease in the growth rate of microorganisms, with the result that it is possible to maintain a high deodorant effect.

Here, the weight ratio (hereinafter the “starch ratio”) R of the starches with respect to the microbial raw material is set to 0.1 to 1.2.

This is because when the starch ratio R is less than 0.1, the nutrient source becomes insufficient such that the growth of the koji mold is not sufficient, and thus the amount of organic acids generated is low such that it is impossible to neutralize highly alkaline ammonia in a short period of time. On the other hand, when the starch ratio R is more than 1.2, although a sufficient amount of starches necessary as the nutrient source for the koji mold can be acquired, a previous extra amount of starches blocks most of the pores so as to prevent the koji mold from being fixed into the pores and thereby reduce the generation of organic acids or the starches thickly cover the surface of the already fixed koji mold so as to prevent contact between the koji mold and ammonia and thereby reduce the neutralization reaction, with the result that it takes much time to neutralize ammonia.

Furthermore, the cylindrical member is formed with a rubber pipe, a plastic pipe, a metal pipe, etc., and the material thereof is not particularly limited as long as the cylindrical member has acid resistance to the organic acids generated as the koji mold is grown and rigidity in which when the cylindrical member is mixed into the main body (hereinafter, referred to as “treatment material main body”) of the deodorant treatment material for tobacco mixed with the carbon raw material, the microbial raw material and the starches, the hole portion (hereinafter, referred to as “cylindrical hole”) of the cylindrical member is prevented from being blocked by the surrounding pressure.

In this way, ventilation is enhanced between the outside and the inside of the treatment material main body and within the treatment material main body, and thus the malodorous components can be spread over the entire treatment material main body, with the result that the physical adsorption and the neutralization reaction described above can be made to efficiently proceed.

More preferably, the starches have the weight ratio of 0.1 to 0.5 with respect to the microbial raw material.

This is because when the starch ratio R is less than 0.1, as described above, the nutrient source becomes insufficient such that the growth of the koji mold is not sufficient, and thus the amount of organic acids generated is low such that it is impossible to neutralize highly alkaline ammonia in a short period of time. On the other hand, when the starch ratio R is between 0.5 and 1.2, unlike the case where the starch ratio R is more than 1.2 described above, the following problems are prevented in which the previous extra amount of starches blocks most of the pores so as to prevent the koji mold from being fixed into the pores or the starches thickly cover the surface of the already fixed koji mold so as to prevent contact between the koji mold and ammonia. However, since the starches begin to block the cylindrical hole of the cylindrical member, the effect of the communication action caused by the cylindrical member which will be described later is lowered, with the result that as compared with the case where the starch ratio R is 0.1 to 05, it takes much time to neutralize ammonia.

Preferably, the microbial raw material contains at least one of the rice koji and the wheat koji.

As compared with the seed koji which is obtained by using for example, a small amount of rice as a raw material so as to separately culture the spore as described above and which is available, when the rice koji or the wheat koji in which a large amount of starches such as rice and wheat is already present is used, it is possible to effectively reduce a decrease in the growth rate of the koji mold using the starches as the nutrient source.

Preferably, the cylindrical member is formed with: a long cylindrical member which is protruded from the inside of the treatment material main body formed with the carbon raw material, the microbial raw material and the starches to the outside; and a short cylindrical member which is shorter than the long cylindrical member and which is embedded in the treatment material main body.

The long cylindrical member in the cylindrical member makes the outside communicate with the inside of the treatment material main body, and by the communication action described above, the malodorous components floating to the outside are passed through a long cylindrical hole and are made to flow to the inside of the treatment material main body, with the result that the physical adsorption and the neutralization reaction are made to efficiently proceed.

Hence, the length of the long cylindrical member is changed depending on the form of use of the deodorant treatment material for tobacco, and the length of the long cylindrical member is preferably set longer than the longest diameter of the treatment material main body at the time of use so that the end portion opening is easily exposed from the treatment material main body.

In this way, even when a small amount of organic acids is generated such as by lack of the starches, it is possible to reduce a time (hereinafter, referred to as “deodorant time”) necessary for decomposing ammonia so as to reduce to a predetermined concentration.

Even when the starches are not sufficiently agitated so as to thickly cover the surface of the treatment material main body, and thus acetaldehyde and formaldehyde floating in the outside are unlikely to reach the surface of the bamboo charcoal within the treatment material main body, acetaldehyde and formaldehyde are efficiently brought into contact with the surface of the bamboo charcoal, with the result that it is possible to reduce the extension of the deodorant time of acetaldehyde and formaldehyde.

Furthermore, the short cylindrical member makes the adjacent koji mold and bamboo charcoal communicate with each other, and by the communication action thereof, the malodorous components entering from the outside into the treatment material main body are passed through a short cylindrical hole and are made to flow between the adjacent koji mold and bamboo charcoal, with the result that the physical adsorption and the neutralization reaction are made to efficiently proceed.

Hence, the length of the short cylindrical member is changed depending on the form of use of the deodorant treatment material for tobacco, and the length of the short cylindrical member is preferably set shorter than the shortest diameter of the treatment material main body so that the end portion opening is prevented from being exposed from the treatment material main body.

In this way, as described above, it is possible to further reduce the deodorant time of ammonia and further improve the deodorant time of acetaldehyde and formaldehyde.

Preferably, the short cylindrical members have an outer circumferential shape of an approximate circle in a cross-sectional view such that when the short cylindrical members are arranged close to each other, a gap can be formed among the outer circumferential surfaces thereof.

The malodorous components entering from the outside into the treatment material main body while being diffused are passed not only through the inside of the cylindrical hole but also through the gap formed between the short cylindrical members arranged close to each other, with the result that it is possible to further enhance the fluidity of the malodorous components between the adjacent koji mold and bamboo charcoal.

In this way, it is possible to still further reduce the deodorant time of ammonia and still further improve the deodorant time of acetaldehyde and formaldehyde.

### Effects of the Invention

The deodorant treatment material for tobacco according to the present invention simultaneously has an excellent deodorant effect on a plurality of malodorous substances generated from tobaccoal though the deodorant treatment material for tobacco has a simple composition.

### BRIEF DESCRIPTION OF THE DRAWINGS

FIG. 1 is an illustrative diagram showing the configuration of a deodorant treatment material for tobacco according to the present invention, FIG. 1(a) is a perspective view of short pipes and FIG. 1(b) is a side view showing the manufacturing conditions of a sample;

FIG. 2 is an illustrative diagram showing a procedure for a deodorant test for the deodorant treatment material for tobacco, FIG. 2(a) is an illustrative diagram showing the conditions of the storage of the sample into a test bag, FIG. 2(b) is an illustrative diagram showing the conditions of the sealing of the sample into the test bag, FIG. 2(c) is an illustrative diagram showing the conditions of the injection of malodorous components and air and FIG. 2(d) is an illustrative diagram showing the conditions of the measurement of the gas concentration of the malodorous components;

FIG. 3 is a graph showing a temporal change in the gas concentration C1 of ammonia;

FIG. 4 is a graph showing the influence of the number of times of tests N exerted on the deodorant necessary time Tb of ammonia;

FIG. 5 is a graph showing the influence of a starch ratio R exerted on the deodorant necessary time Tb10 of ammonia in the tenth test;

FIG. 6 is a graph showing a temporal change in the gas concentration C2 of hydrogen sulfide;

FIG. 7 is a graph showing a temporal change in the gas concentration C5 of acetic acid;

FIG. 8 is a graph showing a temporal change in the gas concentration C6 of pyridine;

FIG. 9 is an illustrative diagram showing the deodorant effect of acetaldehyde, FIG. 9(a) is a graph showing a temporal change in the gas concentration C3 of acetaldehyde and FIG. 9(a) is a graph showing the influence of the starch ratio R exerted on the deodorant necessary time Tb10 of acetaldehyde in the tenth test; and

FIG. 10 is an illustrative diagram showing the deodorant effect of formaldehyde, FIG. 10(a) is a graph showing a temporal change in the gas concentration C4 of formaldehyde and FIG. 10(b) is a graph showing the influence of the starch ratio R exerted on the deodorant necessary time Tb10 of formaldehyde in the tenth test.

### DESCRIPTION OF THE (PREFERRED) EMBODIMENTS

An embodiment of the present invention related to a deodorant treatment material for tobacco will be described below with reference to tables and drawings so that the present invention can be understood.

The deodorant treatment material for tobacco according to the present invention contains a carbon raw material, a microbial raw material, starches which serve as a nutrient source for the microbial raw material and which have a predetermined proportion and cylindrical members which make the carbon raw material and the microbial raw material communicate with the outside.

Among them, the carbon raw material contains bamboo charcoal, and the microbial raw material contains a koji mold which can generate an organic acid.

The koji mold further contains at least one of a rice koji and a wheat koji.

Moreover, as the cylindrical members, long and short cylindrical members are prepared and are mixed with a treatment material main body, the short cylindrical member is formed substantially in the shape of a circle in a cross-sectional view and thus the outside and the inside of the treatment material main body are made to communicate with each other and the adjacent koji mold and bamboo charcoal are made to communicate with each other.

### EXAMPLE

The present invention will be described in detail below by use of an example. However, the present invention is not limited to the example.

[Preparation of Carbon Raw Material, Microbial Raw Material, Starches and Cylindrical Members]

In the present example, bamboo charcoal serving as a carbon raw material was obtained by heating various bamboo such as henon bamboo and Japanese timber bamboo in a carbonization furnace in a nitrogen atmosphere to 600 to 800° C. so as to perform carbonization treatment, and the powdery bamboo charcoal having particle diameters of about 10 μm to about 5 mm was used. A small amount of charcoal added to the bamboo charcoal was obtained by performing, on cedar, the same carbonization treatment as the bamboo charcoal, and the powdery charcoal having the same particle diameters of about 10 μm to about 5 mm was used.

As koji molds serving as a microbial raw material, a koji mold which was obtained by mainly using a powdery rice koji and adding a small amount of seed koji (hereinafter, referred to as “rice koji main raw material”) and a powdery wheat koji alone were used. In the rice koji main raw material, the rice koji and the seed koji were mixed at a weight ratio of 10 to 1.

Powdery starches were obtained by mixing a potato starch and a dogtooth starch at a weight ratio of 1 to 1 (hereinafter, referred to as “mixing starch”), and were used.

For cylindrical members, as a long cylindrical member, a short pipe was used which had a diameter of 5 mm and a length of 15 mm and which was made of polyethylene, and as a short cylindrical member, a long pipe was used which had a diameter of 5 mm and a length of 5 mm and which was made of the same material of polyethylene. The short pipes and the long pipes were mixed at a weight ratio of 4 to 1 (hereinafter, referred to as “mixing pipe”), and were used. Here, the sizes of the individual pipes were set such that in a state of a sample (10 g) used in a deodorant test which will be described later, the end portion opening of the long pipe was protruded from a treatment material main body and that the short pipe was almost embedded within the treatment material main body.

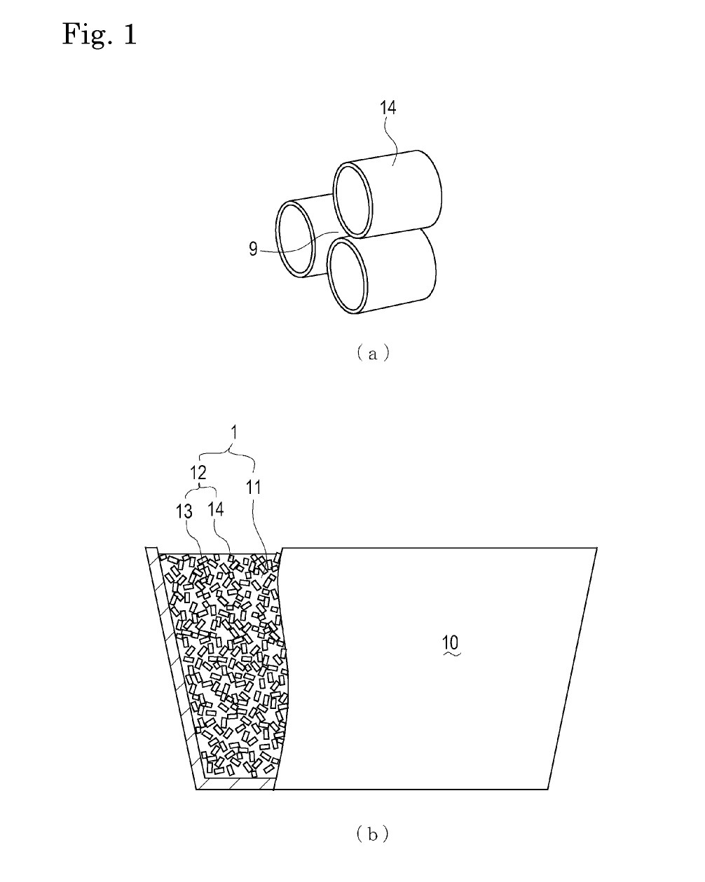

Among them, the short pipes 14 were formed, as shown in FIG. 1(a), substantially in the shape of circles in a cross-sectional view such that a gap 9 can be formed among the outer circumferential surfaces thereof when they were arranged close to each other.

[Manufacturing of Samples]

As shown in FIG. 1(b), the cylindrical members 12 formed with the long pipes 13 and the short pipes 14 were added to the treatment material main body 11 of the prepared carbon raw material, etc., and they were input into a mixing container 10 such that the total amount was about 2.5 kg and were agitated with an unillustrated blade-type agitation device, with the result that the samples of a plurality of types of deodorant treatment materials for tobacco having the following compositions were manufactured.

First, as the carbon raw material, the microbial raw material, the starches and the cylindrical members described above, the bamboo charcoal, the rice koji main raw material, the mixing starch and the mixing pipe were respectively used (hereinafter, referred to as “basic composition system”), and among them, the amount of mixing starch contained was changed from 0.4 to 23.4 wt %, with the result that samples A-1 to A-12 were manufactured.

Furthermore, instead of the rice koji main raw material in the basic composition system described above, a wheat koji was used, and the amount of mixing starch contained was changed from 0.6 to 17.3 wt %, with the result that samples B-1 to B-5 were manufactured.

Moreover, a small amount of charcoal was added to the bamboo charcoal in the basic composition system, and the amount of mixing starch contained was changed from 0.5 to 16.8 wt %, with the result that samples C-1 to C-5 were manufactured.

For the invention materials described above, as a comparative material, a sample X which was obtained by removing the microbial raw material, the starches and the cylindrical members from the basic composition system described above so as to leave only the bamboo charcoal was manufactured.

Furthermore, as comparative materials, the cylindrical members were removed from the basic composition system described above, and the amount of mixing starch contained was changed from 0.4 to 23.4 wt %, with the result that samples Y-1 to Y-12 were manufactured.

Moreover, as comparative materials, instead of the rice koji main raw material in the basic composition system, yeast was used, and the amount of mixing starch contained was changed from 0.6 to 19.7 wt %, with the result that samples Z-1 to Z-5 were manufactured. As the yeast, a powdery baker's yeast was used.

Table 1 shows the compositions of the samples A-1 to A-12, B-1 to B-5 and C-1 to C-5 of the invention materials and the samples X, Y-1 to Y-12 and Z-1 to Z-5 of the comparative materials in the deodorant treatment materials for tobacco manufactured as described above.

TABLE 1

| | | | Composition (wt %) | | | | | |
|---|---|---|---|---|---|---|---|---|
| | | | remaining is carbon raw material | | | | | |
| | | | Microbial raw | | | | Cylindrical | |
| | Sample | Type of carbon | material | | Starches | | member | Starch |
| Class | name | raw material | Type | wt % | Type | wt % | wt % | ratio |
| Invention material | A-1 | Bamboo charcoal | Rice koji main raw material | 11.9 | Mixing starch | 0.4 | Mixing pipe | 0.03 |
| | A-2 | | (small amount of seed koji) | | | 0.7 | (9.2 wt %) | 0.06 |
| | A-3 | | | | | 1.5 | | 0.13 |
| | A-4 | | | | | 3.2 | | 0.27 |
| | A-5 | | | | | 5 | | 0.42 |
| | A-6 | | | | | 6.7 | | 0.56 |
| | A-7 | | | | | 12.5 | | 1.05 |
| | A-8 | | | | | 14.2 | | 1.19 |
| | A-9 | | | | | 15.4 | | 1.29 |
| | A-10 | | | | | 16.9 | | 1.42 |
| | A-11 | | | | | 17.9 | | 1.5 |
| | A-12 | | | | | 23.4 | | 1.97 |
| | B-1 | Bamboo charcoal | Wheat koji | 12.5 | Mixing starch | 0.6 | Mixing pipe | 0.05 |
| | B-2 | | | | | 1.7 | (9.2 wt %) | 0.14 |
| | B-3 | | | | | 5.6 | | 0.45 |
| | B-4 | | | | | 12.3 | | 0.98 |
| | B-5 | | | | | 17.3 | | 1.38 |
| | C-1 | Bamboo charcoal + | Rice koji main raw | 11.9 | Mixing starch | 0.5 | Mixing pipe (9.2 wt %) | 0.04 |
| | C-2 | charcoal | material | | | 2.2 | | 0.18 |
| | C-3 | (8 wt %) | (small amount of seed | | | 4.6 | | 0.39 |
| | C-4 | | koji) | | | 9.7 | | 0.82 |
| | C-5 | | | | | 16.8 | | 1.42 |
| Comparative material | X | Bamboo | — | 0 | — | 0 | — | 0 |
| | | charcoal | | | | | | |
| | Y-1 | Bamboo charcoal | Rice koji main raw material | 11.9 | Mixing starch | 0.4 | — | 0.03 |
| | Y-2 | | (small amount of seed koji) | | | 0.7 | | 0.06 |
| | Y-3 | | | | | 1.5 | | 0.13 |
| | Y-4 | | | | | 3.2 | | 0.27 |
| | Y-5 | | | | | 5 | | 0.42 |
| | Y-6 | | | | | 6.7 | | 0.56 |
| | Y-7 | | | | | 12.5 | | 1.05 |
| | Y-8 | | | | | 14.2 | | 1.19 |
| | Y-9 | | | | | 15.4 | | 1.29 |
| | Y-10 | | | | | 16.9 | | 1.42 |
| | Y-11 | | | | | 17.9 | | 1.5 |
| | Y-12 | | | | | 23.4 | | 1.97 |
| | Z-1 | Bamboo charcoal | Yeast | 13.4 | Mixing starch | 0.6 | Mixing pipe | 0.04 |
| | Z-2 | | | | | 1.9 | (9.2 wt %) | 0.14 |
| | Z-3 | | | | | 6 | | 0.45 |
| | Z-4 | | | | | 11.5 | | 0.86 |
| | Z-5 | | | | | 19.7 | | 1.47 |

[Test Method]

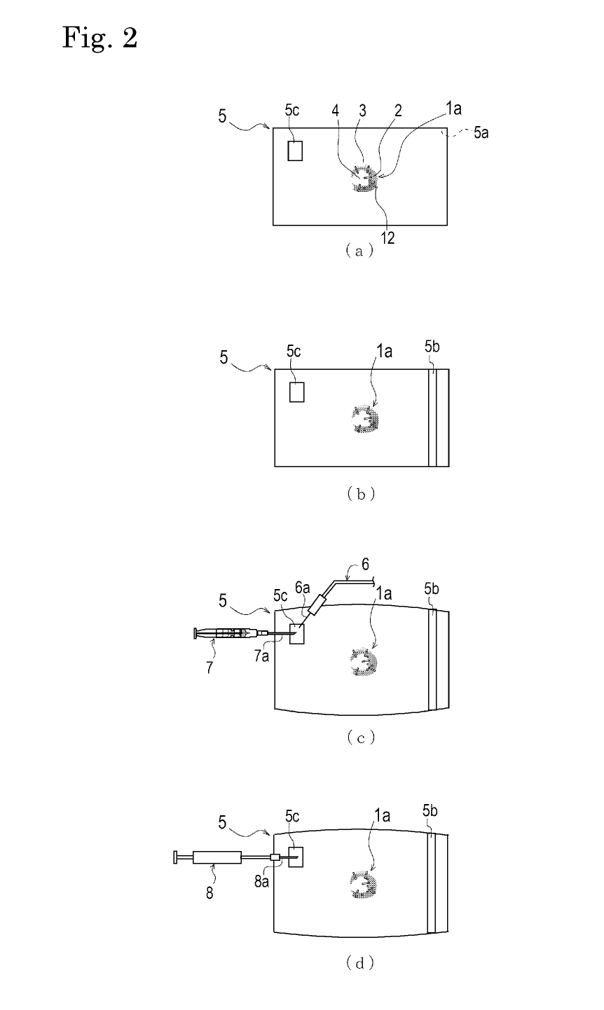

Next, a deodorant test method for these deodorant treatment materials will be described with reference to FIG. 2.

As shown in FIG. 2(a), the sample 1a (10 g) of the deodorant treatment material for tobacco 1 containing the carbon raw material 2, the microbial raw material 3, the starches 4, the cylindrical members 12, etc., is inserted through the opening portion 5a thereof into a sachet 5 whose one end is opened and which is made of transparent polyvinyl fluoride. A rubber plate 5c is adhered to the surface of the sachet 5 on the opposite side to the opening portion 5a, and even if the tip end of a hollow needle is inserted into the sachet 5 so as to penetrate the rubber plate 5c, and thereafter the hollow needle is removed, a needle mark is blocked such that air tightness within the sachet 5 is maintained.

Then, as shown in FIG. 2(b), a heat seal portion 5b is formed parallel to the opening portion 5a by thermal welding so as to hermetically seal the sachet 5, and the sample 1a is sealed into the sachet 5.

Thereafter, as shown in FIG. 2(c), a hollow needle portion 6a provided at one end of a gas tube 6 is made to penetrate the rubber plate Sc described above, and air (9 liters) which is fed by pressure from an unillustrated gas cylinder communicating with the other end of the gas tube 6 is supplied through the needle portion 6a into the sachet 5. At the same time, a hollow needle portion 7a provided at the tip of a syringe 7 is made to penetrate the rubber plate Sc, and the malodorous components of tobacco are injected through the needle portion 7a into the sachet 5.

As the malodorous components of tobacco, ammonia, hydrogen sulfide, acetaldehyde, formaldehyde, acetic acid and pyridine described above were used, and settings were made such that gas concentrations at an early stage of the injection were 100 ppm for ammonia, 20 ppm for hydrogen sulfide, acetaldehyde and formaldehyde, 50 ppm for acetic acid and 10 ppm for pyridine.

Then, as shown in FIG. 2(d), the sachet 5 into which the sample 1a of the deodorant treatment material for tobacco 1 and the malodorous components of tobacco are sealed is left at room temperature for a predetermined time, thereafter a hollow needle portion 8a provided at the tip of a detection tube 8 is made to penetrate the rubber plate 5c and the gas concentrations C1 to C6 of the malodorous components within the sachet 5 are measured. C1, C2, C3, C4, C5 and C6 respectively indicate the gas concentrations (ppm) of ammonia, hydrogen sulfide, acetaldehyde, formaldehyde, acetic acid and pyridine.

[Test Results]

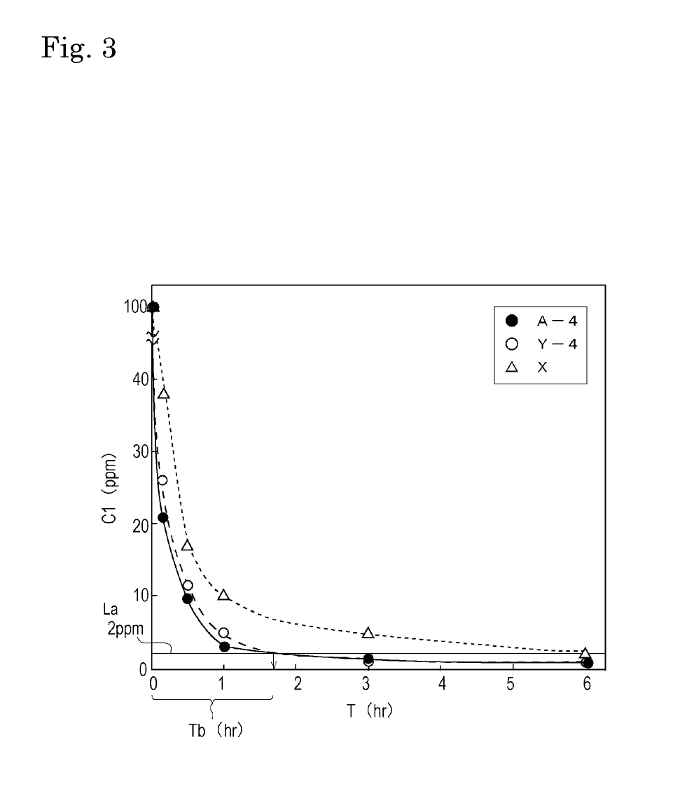

Next, the results of the deodorant test will be described with reference to FIGS. 3 to 10.

1) Ammonia

In Table 2, the results of the measurements of the gas concentrations C1 (ppm) of ammonia within the sachet 5 after ammonia was sealed thereinto for the sample A-4 of the invention sample, the sample Y-4 obtained by removing the cylindrical members from the sample A-4 and the sample X obtained by removing the microbial raw material, the starches and the cylindrical members from the sample A-4 so as to leave only the bamboo charcoal am shown.

TABLE 2

| Elapsed | Gas concentrations | | |
|---|---|---|---|
| time T | C1 of ammonia (ppm) | | |
| (hr) | Sample A-4 | Sample Y-4 | Sample X |
| 0 | 100 | 100 | 100 |
| 0.17 | 21 | 26 | 38 |
| 0.5 | 8 | 12 | 17 |
| 1 | 3 | 5 | 10 |
| 3 | 1 | 1 | 5 |
| 6 | 1 | 1 | 2 |

In Table 2 and FIG. 3, for the sample A-4 of the invention material, the gas concentration C1 was reduced as the time T elapsed after ammonia was sealed thereinto such that the gas concentration was lowered to 2 ppm which was a general regulation reference value La when about 1.7 hours elapsed. By contrast, although for the sample Y-4 of the comparative material, as with the sample A-4, the gas concentration C1 was reduced as the time T elapsed such that the gas concentration was lowered to 2 ppm which was the general regulation reference value La when about 1.7 hours elapsed, the reduction rate of the gas concentration C1 at an early stage of the elapsed time was low as compared with the sample A-4. For the sample X of the comparative material, it took 6 hours for the gas concentration to be lowered to 2 ppm which was the regulation reference value La.

It can be considered that this is because in the sample X, ammonia is adsorbed and removed only by the physical adsorption of the bamboo charcoal whereas each of the samples Y-4 and A-4 contains the rice koji main raw material and the mixing starch, a deodorant effect caused by a neutralization reaction involving the generation of organic acids by the microbial raw material described above is added to the deodorant effect caused by the physical adsorption and thus ammonia is reduced in a short period of time.

Furthermore, it can be considered that in the sample A-4 of the invention material, a communication action caused by the mixing pipe is added, and that as compared with the sample Y-4 of the comparative material, the ability to remove ammonia at an early stage of the elapsed time is enhanced.

With respect to the sample A-4 of the invention material and the sample Y-4 of the comparative material, the same samples were individually used, the deodorant test described above was continuously repeated, temporal changes in the gas concentration C1 were measured for individual rounds and thus from a graph thereof, a time (hereinafter, referred to as “deodorant necessary time”) Tb necessary for lowering the gas concentration C1 to the regulation reference value La (=2 ppm) was determined. In Table 3, the results of the measurements of the deodorant necessary times Tb for the individual numbers of times of tests N are shown.

TABLE 3

| | |
|---|---|
| Number of times | Deodorant necessary |
| of tests N | time Tb (hr) |

| (Number of times) | Sample A-4 | Sample Y-4 |
|---|---|---|
| 1 | 1.7 | 1.7 |
| 2 | 1.7 | 1.6 |
| 3 | 1.5 | 1.8 |
| 4 | 1.6 | 1.9 |
| 5 | 1.6 | 1.9 |
| 6 | 1.7 | 1.8 |
| 7 | 1.4 | 1.6 |
| 8 | 1.5 | 1.8 |
| 9 | 1.5 | 1.9 |
| 10 | 1.3 | 2.1 |

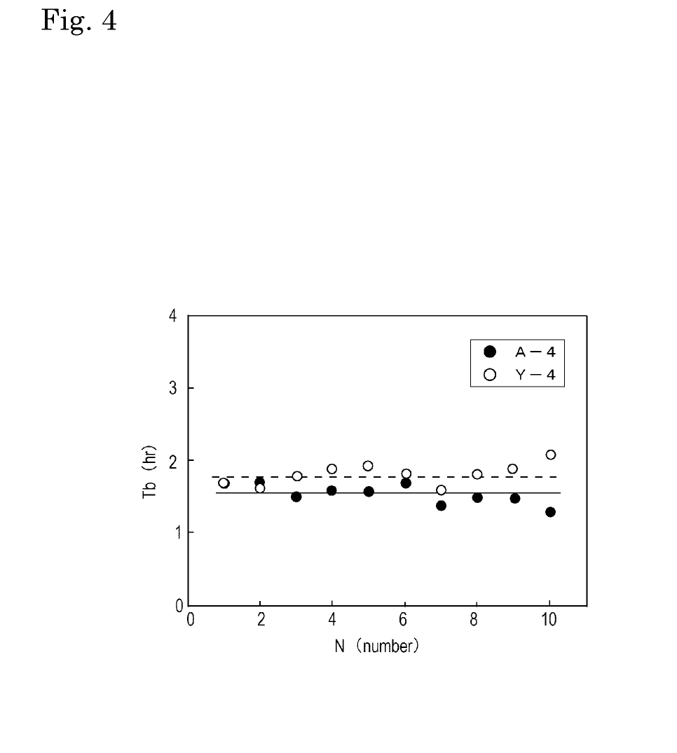

It is found from Table 3 and FIG. 4 that both in the sample A-4 of the invention material and the sample Y-4 of the comparative material, even when the deodorant test was repeated 10 times, the deodorant necessary times Tb were maintained to be short times between 1.4 to 2.1 hours. It can be considered that this is because in each of the samples, the mixing starch is constantly supplied as a nutrient to the rice koji main raw material such that the growth of the koji mold is performed uninterruptedly.

However, there is a tendency that as the number of times of tests N was increased, the deodorant necessary time Tb was shorter in the sample A-4 of the invention material than in the sample Y-4 of the comparative material. It can be considered that this is because in the sample A-4, ammonia is constantly spread over the entire treatment material main body by the effect of the communication action, and thus even when the number of times of tests N is increased, the reaction rate of the neutralization reaction is unlikely to be lowered.

In Table 4, the results of the measurements of deodorant necessary times Tb10 in the tenth test when for the individual types of samples, the deodorant test using the same sample was continuously repeated 10 times are shown.

TABLE 4

| | | | Deodorant necessary time |
|---|---|---|---|
| | Sample | | in the tenth test |
| Class | name | Starch ratio | Tb10 (hr) |
| Invention | A-1 | 0.03 | 3 |
| material | A-2 | 0.06 | 2.5 |
| | A-3 | 0.13 | 1.2 |
| | A-4 | 0.27 | 1.3 |
| | A-5 | 0.42 | 1.1 |
| | A-6 | 0.56 | 1.7 |
| | A-7 | 1.05 | 1.8 |
| | A-8 | 1.19 | 2 |
| | A-9 | 1.29 | 2.2 |
| | A-10 | 1.42 | 2.4 |
| | A-11 | 1.5 | 2.9 |
| | A-12 | 1.97 | 3 |
| | B-1 | 0.05 | 2.6 |
| | B-2 | 0.14 | 1.4 |
| | B-3 | 0.45 | 1.3 |
| | B-4 | 0.98 | 1.7 |
| | B-5 | 1.38 | 2.3 |
| | C-1 | 0.04 | 2.5 |
| | C-2 | 0.18 | 1.5 |
| | C-3 | 0.39 | 1.3 |
| | C-4 | 0.82 | 1.8 |
| | C-5 | 1.42 | 2.6 |
| Comparative | X | 0 | 10.2 |
| material | Y-1 | 0.03 | 4 |
| | Y-2 | 0.06 | 2.8 |
| | Y-3 | 0.13 | 2.1 |
| | Y-4 | 0.27 | 2.1 |
| | Y-5 | 0.42 | 2.2 |
| | Y-6 | 0.56 | 2.3 |
| | Y-7 | 1.05 | 2.1 |
| | Y-8 | 1.19 | 2.2 |
| | Y-9 | 1.29 | 2.4 |
| | Y-10 | 1.42 | 2.7 |
| | Y-11 | 1.5 | 3.3 |
| | Y-12 | 1.97 | 3.6 |
| | Z-1 | 0.04 | 3.1 |
| | Z-2 | 0.14 | 1.6 |
| | Z-3 | 0.45 | 1.8 |
| | Z-4 | 0.86 | 2.1 |
| | Z-5 | 1.47 | 3.2 |

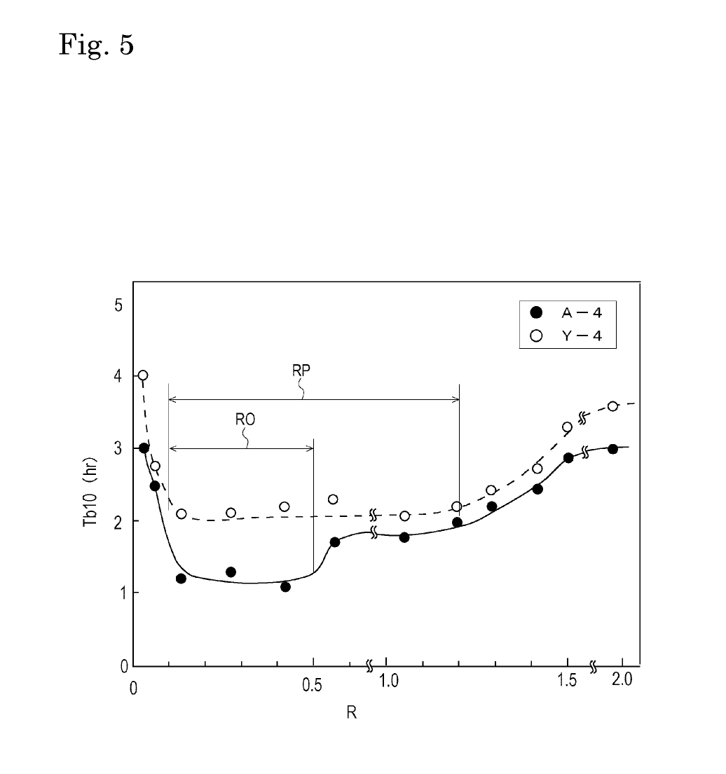

In Table 4 and FIG. 5, the deodorant necessary times Tb10 in the tenth test for the samples A-1 to A-12 in the basic composition system were maintained to be short times between 1.7 and 2.0 hours when a starch ratio R fell within a range of 0.1 to 1.2 (hereinafter, referred to as “appropriate starch ratio range”) RP whereas when the starch ratio R was less than 0.1 or more than 1.2, although the deodorant necessary times Tb10 were shorter than in the sample X in the comparative material, they became longer than the deodorant necessary times Tb10 in the appropriate starch ratio range RP.

Furthermore, specifically, the deodorant necessary times Tb10 were 1.1 to 1.3 hours when the starch ratio R fell within a range of 0.1 to 0.5 (hereinafter, referred to as “optimum starch ratio range”) RO, and were maintained to be particularly short times even within the appropriate starch ratio range RP.

This tendency was substantially the same as in the samples B-1 to B-5 obtained by using the wheat koji instead of the rice koji main raw material and the samples C-1 to C-5 obtained by adding a small amount of charcoal to the bamboo charcoal in the invention material and in the samples Z-1 to Z-5 obtained by using the yeast instead of the rice koji main raw material in the basic composition system in the comparative material.

It can be considered that this is because in the combination of the carbon raw material, the microbial raw material and the starches, although a slight difference is made, when the amount of starches is excessively low, the nutrient source becomes insufficient such that the growth of the microbial raw material is not sufficient whereas when the amount of starches is excessively high, a previous extra amount of starches blocks pores such that the microbial raw material is prevented from being fixed to the carbon raw material or the extra amount of starches covers the already fixed microbial raw material so as to prevent contact with ammonia.

Furthermore, both in the samples B-1 to B-5 and the samples C-1 to C-5 in the invention material, within the appropriate starch ratio range RP, the deodorant necessary times Tb10 which were substantially the same as in the samples A-1 to A-12 in the basic composition system were acquired whereas both in the samples Y-1 to Y-12 and the samples Z-1 to Z-5 in the comparative material, there was a tendency that the deodorant necessary times Tb10 were longer than in the samples A-1 to A-12 in the basic composition system.

It can be considered that this is because in the samples Y-1 to Y-12, the above-described communication action caused by the cylindrical members cannot be obtained, the malodorous components cannot be spread over the entire treatment material main body and thus the reaction rate of the neutralization reaction is lowered. It can be considered that in the samples Z-1 to Z-5, the main nutrient source for the yeast is sugar, and thus a decrease in the growth rate of the yeast cannot be sufficiently reduced by the addition of the starches.

Moreover, the optimum starch ratio range RO described above did not appear in the samples Y-1 to Y-12 obtained by removing the mixing pipe but was recognized in samples having the mixing pipe other than the samples Y-1 to Y-12.

It can be considered that this is because when the amount of starches is increased to a certain extent, the cylindrical hole of the mixing pipe mixed thereinto is blocked by the starches, and thus the effect of the communication action caused by the mixing pipe is lowered. In other words, it can be considered that the presence of the optimum starch ratio range RO even in the appropriate starch ratio range is a specific phenomenon when the mixing pipe serving as the cylindrical member is present.

2) Hydrogen Sulfide, Acetic Acid and Pyridine

For the sample A-4 in the invention material and the samples Y-4 and X in the comparative material, as with ammonia, hydrogen sulfide, acetic acid and pyridine were sealed into the sachet 5, and thereafter changes in the gas concentration were measured.

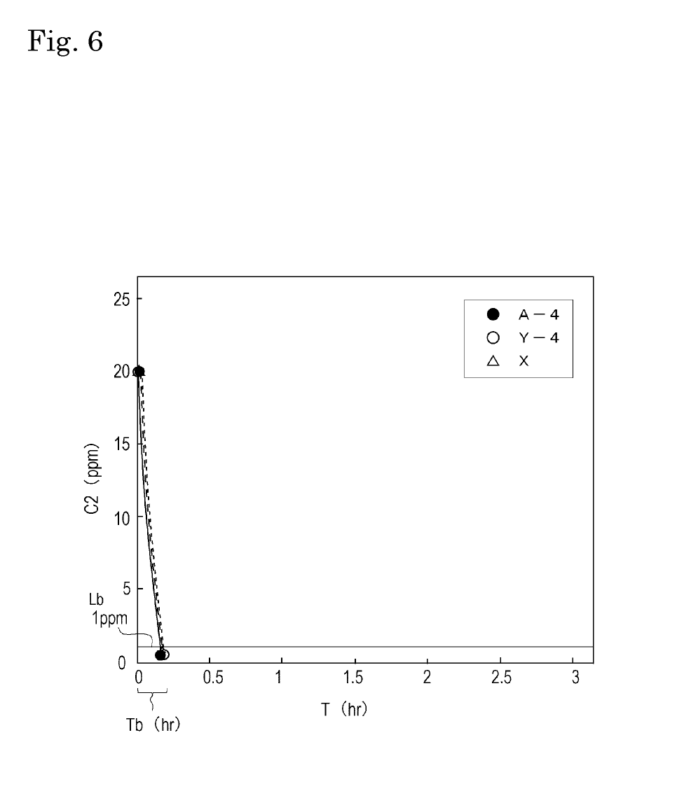

As shown in FIG. 6, in the case of hydrogen sulfide, in any one of the samples A-4, Y-4 and X, as the time T elapsed after hydrogen sulfide was sealed thereinto, the gas concentration C2 thereof was rapidly reduced from 20 ppm a tan early stage of the injection, and was lowered below 1 ppm in only 10 minutes which was the quantitative lower limit value Lb of hydrogen sulfide.

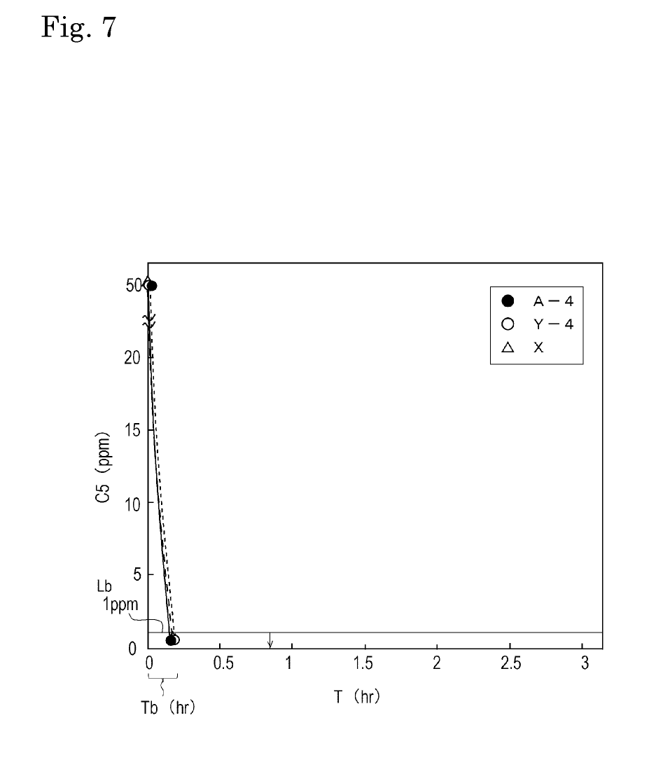

As shown in FIG. 7, in the case of acetic acid as well, in any one of the samples A-4, Y-4 and X, as the time T elapsed after acetic acid was sealed thereinto, the gas concentration C5 thereof was rapidly reduced from 50 ppm at an early stage of the injection, and was lowered below 1 ppm in only 10 minutes which was the quantitative lower limit value Lb of acetic acid.

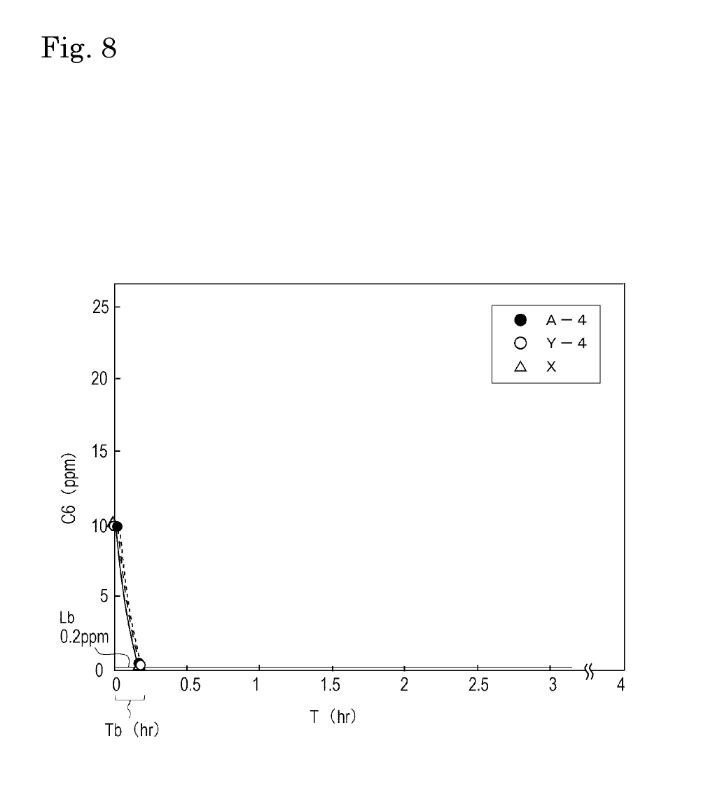

As shown in FIG. 8, in the case of pyridine as well, in any one of the samples A-4, Y-4 and X, as the time T elapsed after pyridine was sealed thereinto, the gas concentration C6 thereof was rapidly reduced from 10 ppm at an early stage of the injection, and was lowered below 0.2 ppm in only 10 minutes which was the quantitative lower limit value Lb of pyridine.

Hence, it is found that hydrogen sulfide, acetic acid and pyridine are sufficiently removed in a short period of time irrespective of the samples. It can be considered that this is because on hydrogen sulfide, acetic acid and pyridine, the deodorant effect caused by the physical adsorption of the bamboo charcoal serving as the component common to the samples A-4, Y-4 and X is significantly exerted. Specifically, it is estimated that this is because, for example, since hydrogen sulfide and acetic acid are acidic, the influence of the neutralization reaction caused by organic acids is unlikely to be exerted, and since pyridine is alkaline as with ammonia but has a high molecular weight, the influence of an intermolecular force which is effective for the physical adsorption is exerted.

3) Acetaldehyde and Formaldehyde

For the sample A-4 in the invention material and the samples Y-4 and X in the comparative material, as with ammonia, acetaldehyde and formaldehyde were sealed into the sachet 5, and thereafter changes in the gas concentration were measured.

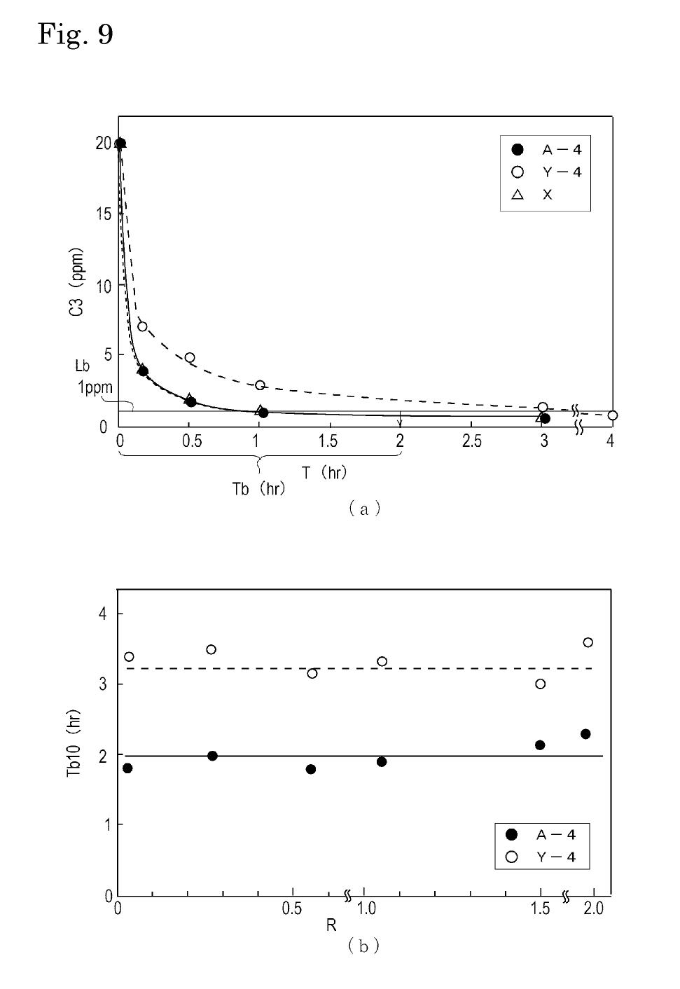

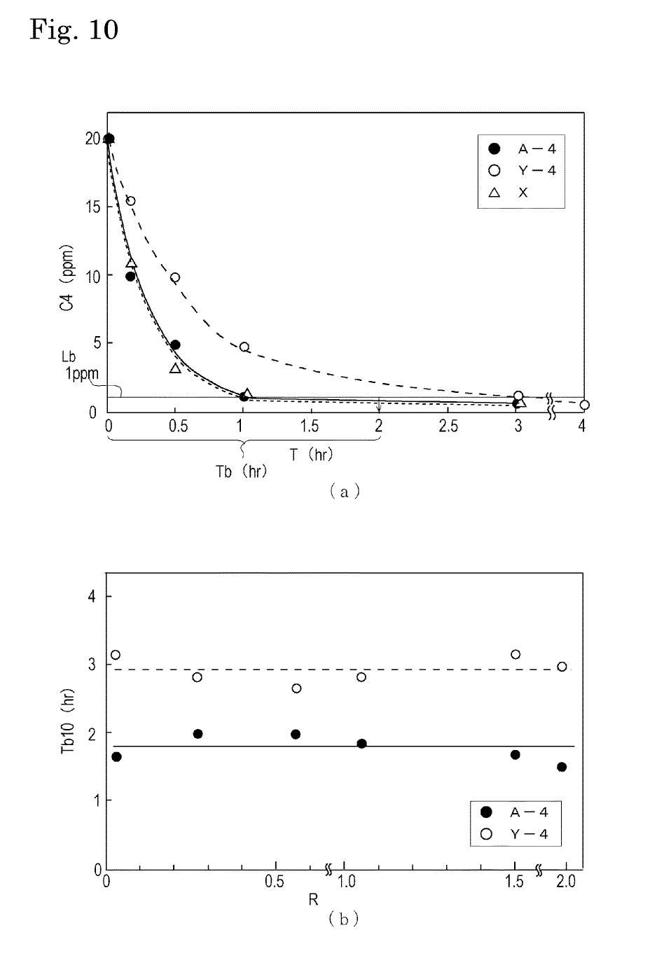

As shown in FIGS. 9(a) and 10(a), in the case of each of acetaldehyde and formaldehyde, in the samples A-4 and X, as the time T elapsed after acetaldehyde was sealed thereinto, the gas concentration C3 thereof was reduced and was lowered below 1 ppm in about 2 hours which was the quantitative lower limit value Lb. By contrast, in the sample X-4, it took 4 hours for the gas concentration C3 to be lowered below 1 ppm which was the quantitative lower limit value Lb.

It can be considered that this is because although in the sample X, the deodorant effect caused by the physical adsorption of the bamboo charcoal is effectively exerted, in the sample Y-4, unlike the sample A-4, the communication action is not present in the mixing pipe, and thus the microbial raw material and the starches cover the bamboo charcoal so as to significantly inhibit the physical adsorption. Specifically, it is estimated that this is because, for example, since acetaldehyde and formaldehyde each are acidic, the influence of the neutralization reaction caused by organic acids is unlikely to be exerted.

Furthermore, as with ammonia, the deodorant necessary times Tb10 in the tenth test when the deodorant test using the same sample was continuously repeated 10 times were measured.

As shown in FIGS. 9(b) and 10(b), in the case of each of acetaldehyde and formaldehyde, the deodorant necessary times Tb10 were a substantially constant value irrespective of the starch ratio R. It can be considered that this is because even in acetaldehyde and formaldehyde, the deodorant effect caused by the physical adsorption of the bamboo charcoal is significantly increased, and that the influence of the addition of the starches is lowered.

Furthermore, in the case of each of acetaldehyde and formaldehyde, the deodorant necessary time Tb10 in the sample A-4 in the invention material was shorter than in the sample Y-4 in the comparative material. It can be considered that this is because even when the number of times of tests N is increased, acetaldehyde and formaldehyde are constantly spread over the entire treatment material main body by the effect of the communication action, and thus the efficiency of the physical adsorption is unlikely to be lowered.

Hence, as described above, in the present invention material, for ammonia, hydrogen sulfide, acetaldehyde, formaldehyde, acetic acid and pyridine which are the malodorous components of tobacco, the physical adsorption caused by the bamboo charcoal and the neutralization reaction caused by the koji mold are made to efficiently proceed by the communication action of the cylindrical member, and thus it is possible to reliably remove all these malodorous components.

As described above, the deodorant treatment material for tobacco to which the present invention is applied simultaneously has an excellent deodorant effect on a plurality of malodorous substances generated from tobaccoal though the deodorant treatment material for tobacco has a simple composition.

### DESCRIPTION OF REFERENCE NUMERALS

1 Deodorant treatment material for tobacco
2 Carbon raw material
3 Microbial raw material
4 Starches
9 Gap
11 Treatment material main body
12 Cylindrical member
13 Long pipe (long cylindrical member)
14 Short pipe (short cylindrical member)
R Starch ratio (weight ratio of starches to microbial raw material)

1. A deodorant treatment material for tobacco comprising:
a highly adsorptive carbon raw material which contains bamboo charcoal;
a microbial raw material which can generate an organic acid and which is formed with a koji mold;
starches whose weight ratio with respect to the microbial raw material is 0.1 to 1.2, and which serve as a nutrient source for the microbial raw material; and
a cylindrical member which makes an outside communicate with the carbon raw material and which makes the outside communicate with the microbial raw material.

2. The deodorant treatment material for tobacco according to claim 1,
wherein the starches have the weight ratio of 0.1 to 0.5 with respect to the microbial raw material.

3. The deodorant treatment material for tobacco according to claim 1,
wherein the microbial raw material contains at least one of a rice koji and a wheat koji.

4. The deodorant treatment material for tobacco according to claim 1,
wherein the cylindrical member is formed with:
a long cylindrical member which is protruded from an inside of a treatment material main body formed with the carbon raw material, the microbial raw material and the starches to the outside; and
a short cylindrical member which is shorter than the long cylindrical member and which is embedded in the treatment material main body.

5. The deodorant treatment material for tobacco according to claim 4,
wherein the short cylindrical members have an outer circumferential shape of an approximate circle in a cross-sectional view such that when the short cylindrical members are arranged close to each other, a gap can be formed among outer circumferential surfaces thereof.
